(19) United States
(12) Patent Application Publication
(10) Pub. No.: US 2024/0268984 A1
(43) Pub. Date: Aug. 15, 2024

(54) HEAD AND ARM RESTRAINT SUPPORT CUFF

(71) Applicants: Herbert W Rock, Plano, TX (US); Denise L. Foster, Plano, TX (US); Mary A. Foster, Plano, TX (US)

(72) Inventors: Herbert W Rock, Plano, TX (US); Denise L. Foster, Plano, TX (US); Mary A. Foster, Plano, TX (US)

(21) Appl. No.: 18/110,087

(22) Filed: Feb. 15, 2023

(51) Int. Cl. A61F 5/37

(52) U.S. Cl. CPC A61F 5/3723

(57) ABSTRACT

A head and arm restraint support cuff for shifting individuals from one location to another is disclosed. The support cuff comprises a head loop or upper large loop connected with one or more arm loops or lower smaller loops for receiving the head and wrist of any individual. Further, the support cuff is an adjustable support cuff. The adjustable support cuff includes a padding with an adjustable strap. The padding further includes one or more insertion clips and pull loops at each end. The adjustable strap is pulled through the insertion clips to increase or decrease the overall length of the padding. Further, one or more receiver clips are attached to the hand loops to accommodate the hands of any individual. The receiver clips are attached facing upwards for receiving the insertion clips. Further, the support cuff is made of any kind of flexible or elastic material.

### TECHNICAL FIELD

The present invention generally relates to an arm support device. More specifically, the present invention relates to a head and arm restraint to provide support to transfer or shift elderly, disabled, hospital patients, or other individuals from one location to another in a caretaking environment.

### BACKGROUND

In a caretaking environment, it is often necessary to shift or transfer elderly or nonambulatory patients from one location to another. The process of shifting patients is accomplished by manually lifting the patients from one location and placing them in another location. Handling an elderly or nonambulatory patient is a serious problem for the caretakers. During transferring patients, some may lose head control, arms control, and others which may result in injuries.

There are various assistive devices to support transferring of elderly or nonambulatory patients from one location to another. Though these devices support the shifting of the patient, they may lack control at times and can result in injuries. Also, the existing assistive devices do not provide comfort to the patients to fold and support their arms during transfer. Further, the caretaker does not have the space for accessing the patient's hands, arms, and neck to check pulse and perform other caretaking functions. Some of them also cause chaffing and other irritation to the body while transferring.

In light of the above-mentioned drawback, there is a need for a head and arm restraint support cuff for safely shifting or transferring an individual from one position to another. Also, there is a need for a head and arm restraint support cuff to prevent the arms of the individual from falling away from the body. Further, there is a need for a head and arm restraint support cuff that enables the individual to comfortably fold and support their arms during transfer. Additionally, there is a need for a head and arm restraint support cuff which is adjustable.

### SUMMARY

The present invention generally discloses an arm support device. More specifically, the present invention discloses a head and arm restraint support cuff to provide support to transfer or shift elderly, disabled, hospital patients, or other individuals from one location to another in a caretaking environment.

In one embodiment, the support cuff is designed as an assistive device worn over the head with arms inserted while shifting individuals. In one embodiment, further the support cuff prevents the arms of an individual from falling away from the body while transferring individuals from one location to another.

In one embodiment, the support cuff comprises a head loop or an upper large loop and one or more arm loops or lower smaller loops. In one embodiment, the head loop and the one or more arm loops are connected forming a head and arm restraint support cuff. In one embodiment, the head loop slides over the head and onto the neck of any individual. In one embodiment, further the one or more arm loops receive the hands or wrist of any individual. In one embodiment, the one or more arm loops are bottom loops. Each arm loop comprises a short strap at their top end. In one embodiment, each arm loops comprises a receiver clip. In one embodiment, the receiver clips are connected to the respective arm loops via their short strap. In one embodiment, the support cuff is constructed of a single strip or a tube of soft fabric stitched to form the upper large loop and two lower small loops large enough to receive the neck and arms of any individual. In one embodiment, further the support cuff is made of any kind of flexible material or elastic material.

In one embodiment, further the adjustable head and arm restraint support cuff includes a padding with an adjustable strap. In one embodiment, the padding along with the adjustable strap forms a head loop to accommodate the neck of any individual. In one embodiment, the padding further includes one or more insertion clips and pull loops attached at each end of the head loop. In one embodiment, the insertion clips are made of plastic or other types of material. In one embodiment, one or more insertion clips and pull loops are attached separately at each end of the padding. In one embodiment, the adjustable support cuff further includes one or more receiver clips for receiving the insertion clips respectively.

In one embodiment, the adjustable strap goes through the insertion clips which are then connected to the pull loops. In one embodiment, the adjustable strap is pulled through the insertion clips to increase or decrease the overall length of the padding. In one embodiment, further the length of the padding is adjustable with the pull loops on each end. In one embodiment, the pull loops are operated after the adjustable support cuff is placed on the patient by simply tugging on them.

In one embodiment, further the support cuff enables the individual to comfortably fold and support his or her arm during a transfer. In one embodiment, further the support cuff allows the caretakers to access hands, arms, and neck of the patient to check pulse and perform other caretaking functions. In one embodiment, the support cuff also allows the caretakers to focus on the safe transfer of an individual rather than arms control. In one embodiment, further the support cuff further avoids chaffing and other irritation of the wrists and neck of the patient.

The above summary contains simplifications, generalizations, and omissions of detail and is not intended as a comprehensive description of the claimed subject matter but, rather, is intended to provide a brief overview of some of the functionality associated therewith. Other systems, methods, functionality, features, and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 shows a perspective view of a head and arm restraint support cuff in an embodiment of the present invention.

FIG. 2 shows a padding with an adjustable strap of the head and arm restraint support cuff in one embodiment of the present invention.

FIG. 3 shows a perspective view of the arm restraint support cuff in one embodiment of the present invention.

FIGS. 4-5 show various perspective views of an insertion clip of the head and arm restraint support cuff in one embodiment of the present invention.

FIG. 6 shows the arm restraint support cuff configured to connect to a padding in one embodiment of the present invention.

FIGS. 7-8 show various perspective views of an individual wearing the head and arm restraint support cuff in one embodiment of the present invention.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

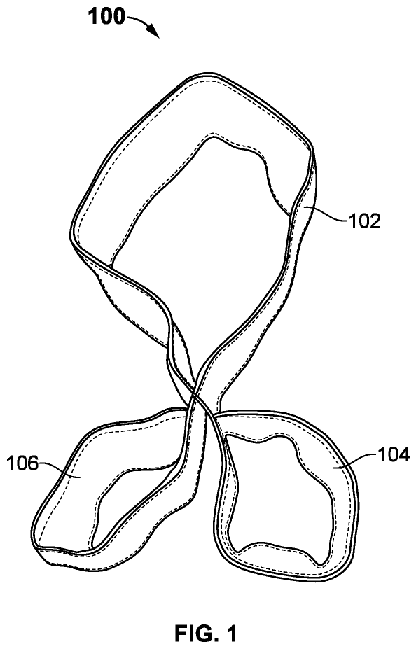

Referring to FIG. 1, a perspective view of a head and arm restraint support cuff 100 is illustrated. In one embodiment, the head and arm restraint support cuff (hereafter referred as “support cuff”) 100 is designed for safely transferring or shifting elderly, disabled, hospital patients, and other individuals from one position or furniture to another in a caretaking environment (i.e., a bed to chair). In one embodiment, the support cuff 100 is used as an assistive device worn over the head with arms inserted while shifting the individuals. In one embodiment, the support cuff 100 prevents the arms of the individual from falling away from the body while transferring them from one location to another.

In one embodiment, the support cuff 100 comprises a head loop or an upper large loop 102 and one or more arm loops or lower smaller loops (104 and 106). In one embodiment, the head loop 102 and the one or more arm loops (104 and 106) are connected to form the head and arm restraint support cuff 100. The head loop 102 slides over or slips around the head and onto the neck of the individual. The one or more arm loops (104 and 106) receive the hands or wrist of the individual. In one embodiment, the support cuff 100 is constructed of a single strip or a tube of soft fabric stitched to form the upper large loop 102 and two lower small loops (104 and 106). The loops (102, 104, and 106) are large enough to receive the neck and arms of any individual. In one embodiment, the support cuff 100 is made of any kind of flexible material or elastic material.

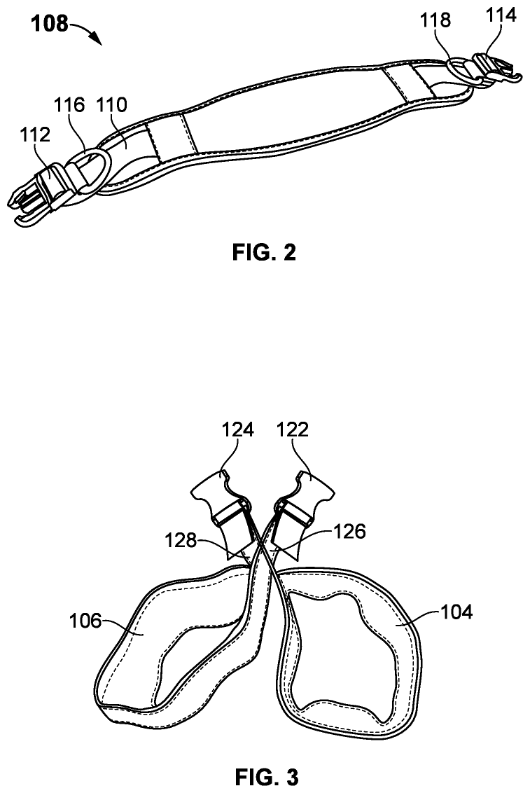

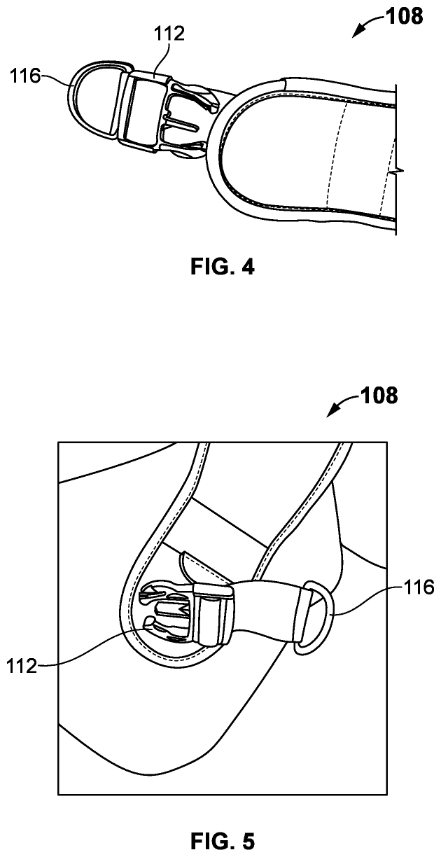

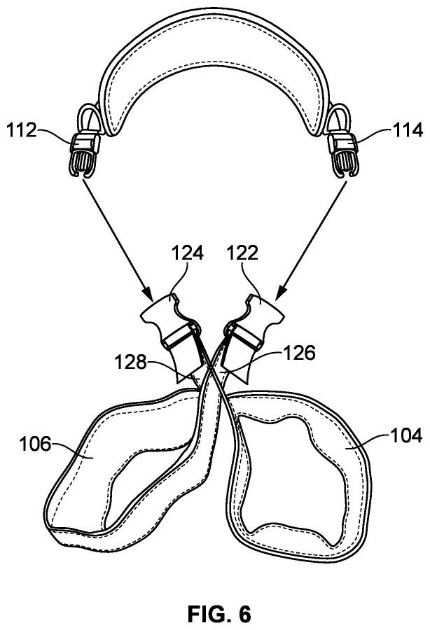

Referring to FIGS. 2-6, one or more components of the head and arm restraint support cuff 100 are illustrated. In one embodiment, the support cuff 100 is an adjustable head and arm restraint support cuff or adjustable support cuff. In one embodiment, the support cuff 100 comprises a padding 108 having an adjustable strap 110 (as shown in FIG. 2). In one embodiment, the one or more arm loops (104 and 106) of support cuff 100 are bottom loops. Each arm loop (104 and 106) further comprises a short strap (126 and 128) at their top end, respectively. In one embodiment, each arm loops (104 and 106) comprises a receiver clip (122 and 124), respectively. In one embodiment, the receiver clips (122 and 124) are connected to the respective arm loops (104 and 106) via their short strap (126 and as click 128) (as shown in FIG. 3). In one embodiment, the padding 108 along with adjustable strap forms the head loop 102 to accommodate the neck of any individual. In one embodiment, the padding 108 further includes one or more insertion clips including a first insertion clip 112 and a second insertion clip 114. In one embodiment, the insertion clips (112 and 114) are attached at each end of the head loop (as shown in FIGS. 2 and 4) 102. In one embodiment, the insertion clips (112 and 114) are made of plastic or other types of material. In one embodiment, the receiver clips (122 and 124) are upwardly connected to the ends of the short straps (126 and 128) of the arm loops (104 and 106) configured to receive one or more insertion clips (112 and 114), thereby creating an adjustable arm restraint.

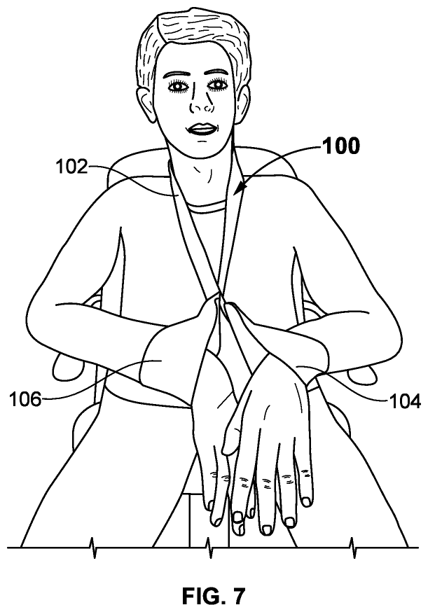

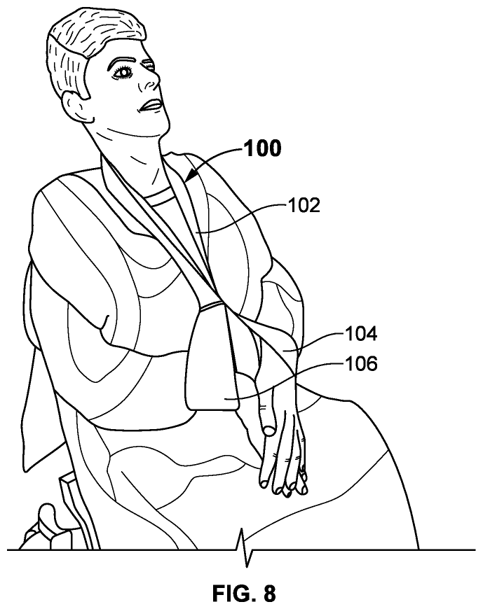

In one embodiment, the padding 108 further includes one or more pull loops (116 and 118) attached at each end of the head loop (as shown in FIGS. 2 and 4) proximal to the insertion clips (112 and 114). In one embodiment, the one or more insertion clips (112 and 114) and pull loops (116 and 118) are attached separately at each end of the padding 108 (as shown in FIG. 5). In one embodiment, the support cuff 100 further comprises one or more receiver clips (122 and 124). The insertion clips (112 and 114) of the padding 108 are configured to attach to the receiver clips (122 and 124) of the arm loops (104 and 106) configured to form an adjustable head and arm restraint (as shown in FIG. 6). In one embodiment, the receiver clips (122 and 124) are attached to the arm loops (104 and 106) configured to accommodate the hands of the individual (as shown in FIGS. 7 and 8). In one embodiment, the receiver clips (122 and 124) are attached facing upwards for receiving the insertion clips (112 and 114) respectively.

In one embodiment, the adjustable strap 110 is stitched along the length of the padding 108. The adjustable strap 110 has at least two ends including a first end and a second end. The first end of the adjustable strap 110 is connected to the first insertion clip 112, whereas the second end is connected to the second insertion clip 114. In one embodiment, the adjustable strap 110 is pulled through the insertion clips (112 and 114) to increase or decrease the overall length of the padding 108. In one embodiment, further the length of the padding 108 is adjustable with the pull loops (116 and 118) on each end. In one embodiment, the pull loops (116 and 118) are operated after the adjustable support cuff 100 is placed on the patient by simply tugging on them.

Referring to FIGS. 7-8, the head and arm restraint support cuff 100 worn by the individual in a typical use case is illustrated. In one embodiment, the head loop 102 slips over the neck of the individual, and one or more arm loops (104 and 106) are inserted with the individual's arm (as shown in FIGS. 7 and 8). In one embodiment, the support cuff 100 prevents the arms of the individual from falling away from the body and the injuries which may result from lack of control. In one embodiment, the support cuff 100 enables the individual to comfortably fold and support their arms during the transfer.

In one embodiment, the caretakers have access to hands, arms, and neck to check pulse and perform other caretaking functions. In one embodiment, the support cuff 100 allows caretakers to focus on the safe transfer of the individual rather than arms control. Further, the support cuff 100 avoids chaffing and other irritation of the wrists and neck of the patient.

Advantageously, the head and arm restraint support cuff 100 of the present invention is simple to construct and easy to use. The support cuff 100 is washable and reusable. The support cuff 100 further improves the safety of the individual while transferring from one position to another. The support cuff 100 prevents injury of the individual while transferring from one location to another in a caretaking environment.

While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The description of the present disclosure has been presented for purposes of illustration and description, but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

1) An arm support device to support a head and an arm of a user, comprising: a head and arm restraint support cuff configured to provide support to transfer the user from a first position to a second position; configured to be worn over the head and the arms of the user and configured to prevent the arms from falling away from a body of the user;
wherein the arm restraint support cuff includes a head loop to be worn over the head of the user and an arm loop connected to the head loop and to be worn over the arm of the user; wherein the head loop is configured to be larger than the arm loop.

2) An arm support device to support a head and an arm of a user as in claim 1, wherein the head loop is configured to slide over the head and onto a neck of the user.

3) An arm support device to support a head and an arm of a user as in claim 1, wherein the arm loop is configured to receive a wrist of the user.

4) An arm support device to support a head and an arm of a user as in claim 1, wherein the head loop and the arm loop are formed from a single strip of fabric.

5) An arm support device to support a head and an arm of a user as in claim 1, wherein the head loop and the arm loop are formed from a single tube of flexible fabric.

6) An arm support device to support a head and an arm of a user as in claim 1, wherein the head loop includes padding with an adjustable strap.

7) An arm support device to support a head and an arm of a user as in claim 6, wherein the head loop includes an insertion clip.

8) An arm support device to support a head and an arm of a user as in claim 6, wherein the head loop includes a pull loop attached at each end of the head loop configured to adjust the size of the head loop.

9) An arm support device to support a head and an arm of a user as in claim 7, wherein the support cuff includes a receiver clip for receiving the insertion clip.

10) An arm support device to support a head and an arm of a user as in claim 7, wherein the support cuff includes a pull loop configured to adjust the size of the head loop.
